(19) United States
(12) Patent Application Publication
(10) Pub. No.: US 2026/0042997 A1
(43) Pub. Date: Feb. 12, 2026

(54) CARBON SEQUESTRATION METHOD AND APPARATUS

(71) Applicant: Regenerative Solutions Global LLC, Graceville, FL (US)

(72) Inventors: Nathaniel BURTON, Graceville, FL (US); Rebecca BURTON, Graceville, FL (US); Phillip BURTON, Graceville, FL (US); Michael AYERS, Inlet Beach, FL (US)

(73) Assignee: Regenerative Solutions Global LLC, Graceville, FL (US)

(21) Appl. No.: 19/296,192

(22) Filed: Aug. 11, 2025

Related U.S. Application Data
(60) Provisional application No. 63/681,344, filed on Aug. 9, 2024.

(51) Int. Cl. C12N 1/14; C05F 11/08; C12R 1/645

(52) U.S. Cl. CPC C12N 1/14; C05F 11/08; C12R 2001/645

(57) ABSTRACT

A method and apparatus for carbon sequestration includes multiple steps, including creating a fungi-rich compost. Next, one extracts the fungi from the compost, such as by liquid extraction. In a subsequent step, one transports the extract to an application site, such as a pasture. And in another subsequent step, water and nutrients are added to the extract to greatly increase the quantity of fungi. This converts the extract into a sort of fortified “tea”, quite rich in fungi and microbes. Next, the resulting “tea” is applied to the soil at the application site to facilitate the growth of fungi in the soil. This has the effect of taking carbon from the atmosphere and placing the carbon in the growing fungi, thereby sequestering atmospheric carbon in fungi.

### BACKGROUND

Carbon dioxide is a primary greenhouse gas widely considered to be contributing to climate change. Carbon sequestration, generally speaking, is the capture of carbon from carbon dioxide and storing it in liquid or solid form, thus preventing it from accumulating further in the atmosphere. Given the widely reported climactic changes in recent years and those changes being attributable at least in part to carbon dioxide in the atmosphere, there is a growing interest in carbon sequestration.

Accordingly, it can be seen that there exists a need for new and effective methods and apparatuses for capturing and sequestering large volumes of carbon in a solid or a liquid state. It is to the provision of such that the present invention is primarily directed.

### SUMMARY OF THE INVENTION

Briefly described, in a first example form the present invention relates generally to a method and apparatus for sequestration of carbon. In one example form, the method includes multiple steps. The first step according to the example comprises creating a fungi-rich compost. Next, one extracts the fungi from the compost, such as by liquid extraction. In a subsequent step, one transports the extract to an application site, such as a pasture. And in another subsequent step, water and nutrients are added to the extract to greatly increase the quantity of fungi. This converts the extract into a sort of fortified “tea”, quite rich in fungi and microbes. Next, the resulting “tea” is applied to the soil at the application site to facilitate the growth of fungi in the soil. This has the effect of taking carbon from the atmosphere (even if in the soil) and placing the carbon in the growing fungi, thereby sequestering atmospheric carbon in fungi.

Optionally, the step of extracting the fungi from the compost can be carried out in a liquid extraction process. The process can involve filling brewing bags with fungi-rich compost and then placing a first filled brewing bag in water for a time. The fungi can be separated from the compost and then the first brewing bag can be removed from the water. The second filled brewing bag is placed in the water for a time, then the fungi are separated from the compost in the second brewing bag, and then the second brewing bag is removed from the water. In short, fungi dislodged from the compost while in the brewing bags while in the water places the fungi in the water even after the brewing bags are removed from the water. In this way a sort of “tea” is brewed from the bags containing the fungi and compost. This tea is heavily laden with fungi and microbes.

The fungi can be separated from the compost mechanically. This mechanical step can comprise agitating the compost with high velocity water or high velocity air or other means. For example, the step of extracting fungi can include mechanically separating fungi from the compost by impinging the compost with pressurized water or high-speed flowing water. It can also be accomplished by vibrating the compost. Alternatively, the step of extracting fungi can include mechanically separating fungi from the compost ultrasonically.

The method can include that the step of extracting fungi comprises filling brewing bags with fungi-rich compost, placing a first filled brewing bag in water for a time, then separating the fungi from the compost, and then the first brewing bag can be removed from the water. Optionally, a second compost-filled brewing bag can be provided, and then placed in the water for a time, and the fungi from the compost in the second brewing bag can be separated from the compost, and then the second brewing bag can be removed from the water.

The generally discontinuous process described above is one way of carrying out the method. Another way is to carry out these method steps in a more or less continuous fashion. In a continuous method, brewing bags containing the compost can be lowered into and raised out of a flowing stream of water, with a residence time in the flowing water selected to allow for the fungi and microbes to be dislodged from the compost and become entrained in the flowing water.

In another example form, the present invention relates to an apparatus for creating a fungi-rich tea to be applied to a soil site for sequestration of carbon into the soil. The apparatus includes conduit for receiving/containing a flowing water stream and another apparatus for adding compost to the flowing water stream at a first position thereof. At a second position in the water stream an apparatus is provided for removing compost from the water stream. In between the two positions in which the compost is added and removed, a mechanical separator is positioned for separating the fungi from the compost. The mechanical separator can take the form of a water jet or an air jet or a vibratory element or other such mechanical device for knocking the fungi loose from the compost to create a fungal-rich extract. The conduit includes an outlet for receiving the fungi-rich extract and delivering it to storage tanks or delivery trucks.

### BRIEF DESCRIPTION OF THE DRAWING FIGURES

FIG. 1 is a schematic illustration of a method for carbon sequestration according to a first example form of the present invention, showing a number of process steps.

FIG. 2A is a schematic illustration of the method for carbon sequestration of FIG. 1, and showing a number of process steps that are part of one of the steps of FIG. 1 in an optional form.

FIG. 2B is a schematic illustration of the method for carbon sequestration of FIG. 1, and showing a number of process steps that are part of one of the steps of FIG. 1 in another optional form.

FIG. 3 is a schematic illustration of an apparatus for carrying out the method for carbon sequestration of FIG. 1, and depicting a steady state or continuous process being performed by the apparatus.

FIG. 4 is a schematic illustration of a method for carbon sequestration according to another example form of the present invention, showing a number of process steps.

### DETAILED DESCRIPTION OF EXAMPLE EMBODIMENTS

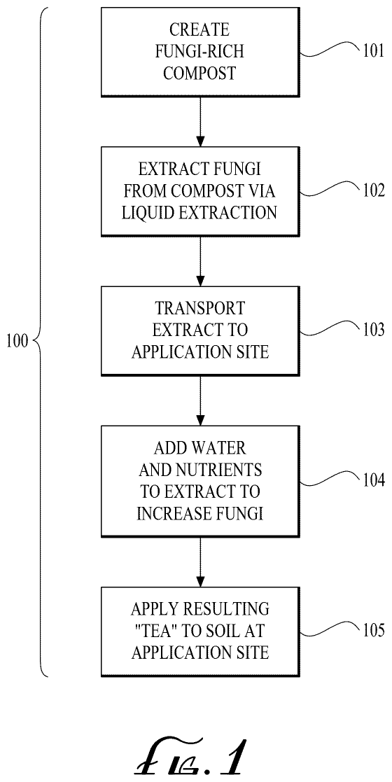

Generally described, the present invention relates to a method and apparatus for carbon sequestration. In one example form as depicted in FIG. 1, the method 100 includes multiple steps. The first step 101 according to the example comprises creating a fungi-rich compost. Next, at step 102 one extracts the fungi from the compost, such as by liquid extraction. In a subsequent step 103, one transports the extract to an application site, such as a pasture. And in another subsequent step 104, water and nutrients are added to the extract to greatly increase the quantity of fungi. This converts the extract into a sort of fortified “tea”, quite rich in fungi and microbes. Next, in step 105 the resulting “tea” is applied to the soil at the application site to facilitate the growth of fungi in the soil. This has the effect of taking carbon from the atmosphere (even if in the soil) and placing the carbon in the growing fungi, thereby sequestering atmospheric carbon in fungi. Optionally, the sequence of steps 103 and 104 can be reversed.

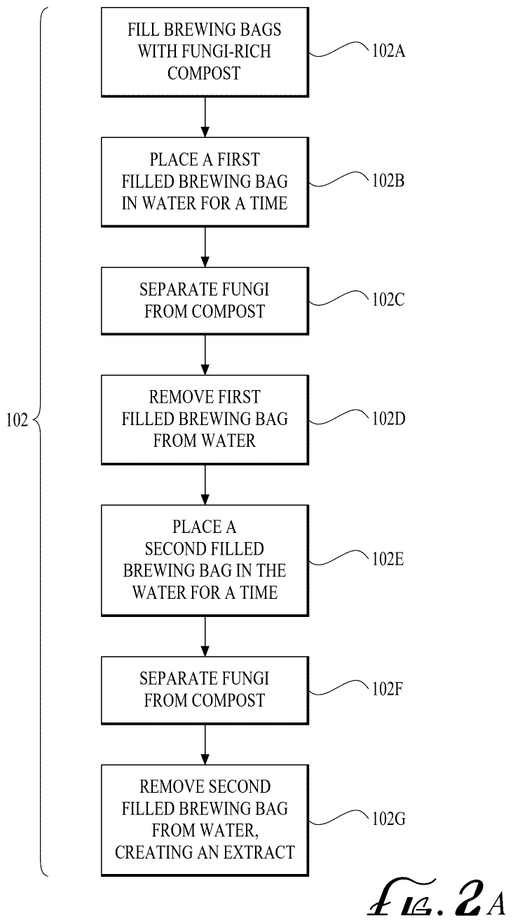

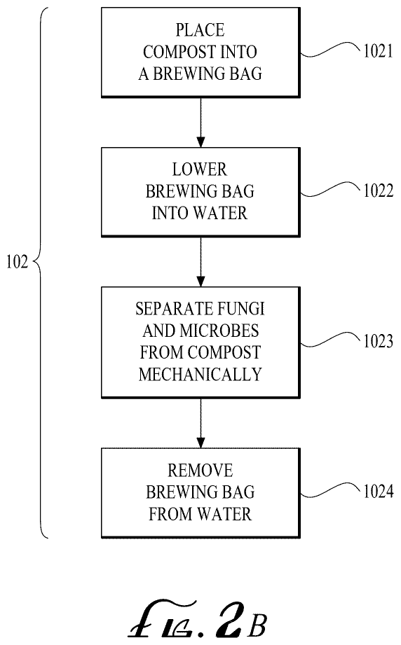

FIG. 2A is a schematic illustration of the method for carbon sequestration of FIG. 1, and showing a number of process steps that are part step 102 of FIG. 1 in an optional form. Optionally, the step 102 of extracting the fungi from the compost can be carried out in a liquid extraction process. The process can involve a first step 102A of filling brewing bags with fungi-rich compost and then a step 102B of placing a first filled brewing bag in water for a time. The fungi are then separated from the compost in step 102C and then the first brewing bag is removed from the water in step 102D. In step 102E, another filled brewing bag is placed in the water for a time and then in step 102F the fungi are separated from the compost in the second brewing bag. Next, in step 102G the second brewing bag is removed from the water. In short, the fungi are dislodged from the compost while the brewing bags are in the water. The fungi remain in the water even after the brewing bags are removed from the water. In this way a sort of fungi-rich “tea” is brewed from the bags containing the fungi and compost. This tea is heavily laden with fungi and microbes.

The fungi can be separated from the compost mechanically. This mechanical step (102C, 102F) can comprise agitating the compost with high velocity water or high velocity air or other means. For example, the step of extracting fungi can include mechanically separating fungi from the compost by impinging the compost with pressurized water or high-speed flowing water or air. It can also be accomplished by vibrating the compost. Alternatively, the step of extracting fungi can include mechanically separating fungi from the compost ultrasonically. Other methods can be employed also.

As shown in FIG. 2B, the method 100 can include that the step 102 of extracting fungi comprises filling brewing bags with fungi-rich compost in step 1021, placing a first filled brewing bag in water for a time in step 1022, then separating the fungi from the compost in step 1023, and then the first brewing bag can be removed from the water in step 1024. Optionally, this sub-process (steps 1021-1024) can be repeated for a second compost brewing bag. Thus, a second compost-filled brewing bag can be provided, and then placed in the water for a time, and the fungi from the compost in the second brewing bag can be separated from the compost, and then the second brewing bag can be removed from the water. While this describes processing one bag at a time, those skilled in the art will appreciate that this method can be scaled up to handle multiple bags at once.

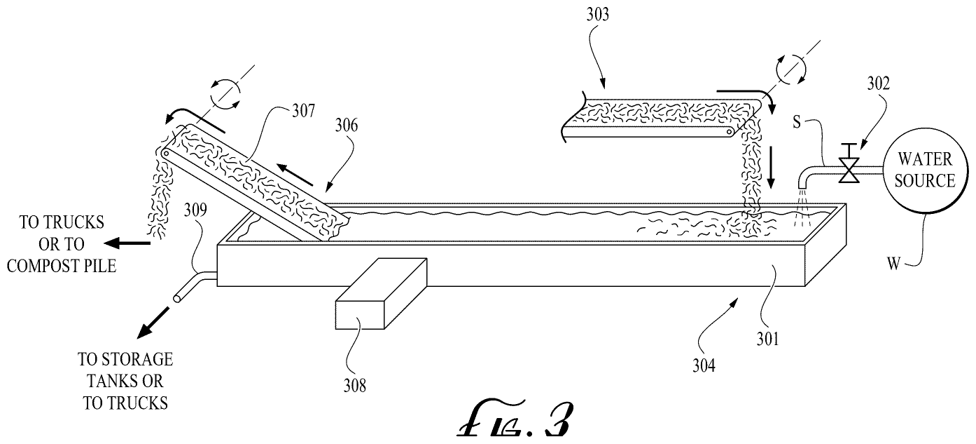

FIG. 3 is a schematic illustration of an apparatus 300 for carrying out the method for carbon sequestration of FIG. 1, and depicts a more or less steady-state or continuous process being performed by the apparatus 300. In this example form, the apparatus 300 is provided for creating a fungi-rich tea to be applied to a soil site for sequestration of carbon into the soil. The apparatus 300 includes a long, wide conduit 301 for receiving and/or containing a flowing water stream S provided from a water source W and controlled by an inlet valve 302. A mechanism 303 is provided for adding compost to the flowing water stream at a first position 304 thereof. The mechanism 303 depicted in the figure is a conveyor, but other mechanisms can be utilized. At a second position 306 in the water stream, another conveyor mechanism 307 is provided for removing compost from the water stream. Between the positions 304, 306 (in which the compost is added and removed), a mechanical separator 308 is positioned for separating the fungi from the compost to create a fungal-rich extract. The mechanical separator 308 can take the form of a water jet or an air jet or a vibratory element or other such mechanical device for knocking the fungi loose from the compost. The conduit includes an outlet 309 for receiving the fungi-rich extract and delivering it to storage tanks or delivery trucks. Also, the conveyor 307 can deliver the removed compost to waiting trucks and/or to a compost pile. The fungal-rich extract can be augmented with additional water and/or nutrients.

The inventors have found that the composition of the compost can have a big impact on producing fungal-dominant/fungi-rich compost. The inventors have used multi-year aged static piles (mother piles) to start new piles (finish piles). These aged piles have been made of ramial wood chips and various sources of high nitrogen, including fish waste from local fish markets and manure from cattle and chickens. This worked rather well. Moreover, spent brewer's grain has worked surprisingly well as a source of nitrogen in producing the finish compost piles. The levels of fungal biomass measured was significantly higher than anything the inventors had measured before. Optionally, one can use vermicompost as the compost.

The bags are filter bags and function like enormous “tea bags”. A mesh size of 400 micron has been found to work well. The batch size in each bag can be of various sizes. Experiments with 10 gallons of compost for a batch of 275 gallons of concentrated extract have yielded excellent results. Regarding the process time or residence time, it has been found that running two cycles to produce the concentrate works quite well. Optionally, the cycles can be 30 minutes each. More preferably, two 20-minute cycles have been found to work well. After the first cycle, the compost tea bags are removed (typically two such bags), and replaced with new tea bags in the brewer filled with fresh compost, and then a second 30-minute cycle is begun. A truck-bed crane can be adapted to the brewer for lifting and lowering the bags down to the precise level in the water.

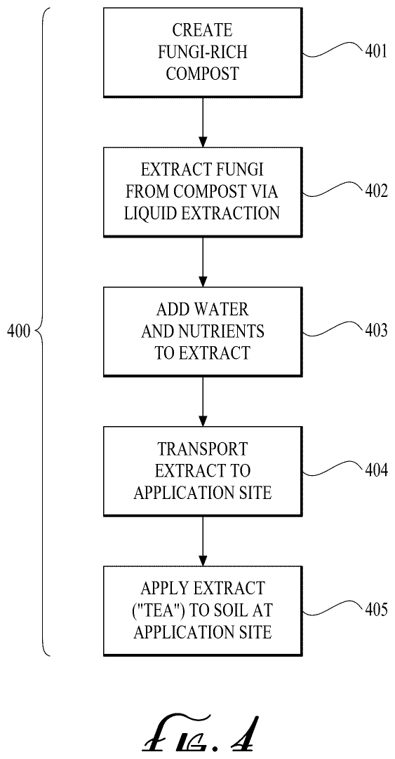

In the method shown in FIG. 1, the extract is transported to the application site and then is mixed with water and nutrients. Transporting the extract before adding water reduces the volume of liquid to be stored and transported prior to application. It is possible, however, to reverse the sequence and add the nutrients before transporting the extract to the application site. This is what is shown in FIG. 4. As shown in this figure, the first step 401 of the method 400 is to create a fungi-rich compost. Next, at step 402 one extracts the fungi from the compost, such as by liquid extraction. In a subsequent step 403, water and nutrients are added to the extract to greatly increase the quantity of fungi. This converts the extract into a sort of fortified “tea”, quite rich in fungi and microbes. Then, one transports the fortified or enhanced extract to an application site, such as a pasture, in step 404, Finally, in step 405 the resulting “tea” is applied to the soil at the application site.

The inventors have found that adding an equal amount of water to the extract yields excellent results. Thus, for a 275 gallon batch of concentrate, we are able to mix up a 550 gallon solution of extract (enhanced or fortified “tea) by adding 275 gallons of water. Ideally, one would apply the “foods” or nutrients only at the time of application at the soil site, or within a very short time window prior to application (a few hours). Doing this at or near to the time of application helps prevent the solution from becoming anaerobic. Preferably, the foods or nutrients selected for successfully propagating the fungi include organic oats and/or organic sea kelp. These products preferably are milled so that they are easily solubilized and are each applied at a rate of 1 cup per 275 gallons of enhanced/fortified extract solution (the end product). Optionally, one can add additional enhancers. For example, one can add Sea 90 Mineral Salt, optionally at a rate of 1 cup per 275 gallons of liquid. Also, one can add Pacific Gro Oceanic Hydrolosate from AgriBio Systems at a rate of 1 quart per 275 gallons of liquid (Pacific Gro Oceanic Hydrolosate contains ground and enzymatically digested fish, shrimp, and crab, high in fish oil).

The carbon being captured and sequestered in the soil is sourced from the air (atmosphere). This is accomplished through the plant root zone.

Recent field tests have shown an increase of 3600% in fungal biomass and the volumes of fungal biomass are so high as to be an effective means of sequestration of carbon in the soil.

In the present disclosure, those skilled in the art will appreciate that one can create the fungal-rich extract using “tea bags” of compost or using loose compost, both being steeped in water. Also, the process can be a batch process or a continuous process. Further, the particular apparatus disclosed herein is an example and those skilled in the art will understand that various modifications to the example apparatus can be made. For example, while a linear water vessel is shown, any shape can be employed as desired. Further, while conveyors are shown and described for introducing and removing compost, other devices can be employed to introduce and remove the compost from the water.

It is to be understood that this invention is not limited to the specific devices, methods, conditions, and/or parameters described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the specific sequence of steps described but can be carried out in other sequences, unless expressly stated otherwise herein.

While the invention has been shown and described in exemplary forms, it will be apparent to those skilled in the art that many modifications, additions, and deletions can be made therein without departing from the spirit and scope of the invention as defined by the following claims.

What is claimed is:

1. A method for sequestration of carbon, comprising the steps of:
creating a fungi-rich vegetative compost;
placing the fungi-rich vegetative compost in liquid;
extracting fungi from the compost to create a liquid extract;
adding water and nutrients to the liquid extract to greatly increase the quantity of fungi in the liquid extract to create a fungi-rich extract; and
applying the fungi-rich extract to the soil at an application site to facilitate the growth of fungi in the soil.

2. The method of claim 1 wherein the step of extracting fungi comprises mechanically separating fungi from the compost.

3. The method of claim 1 wherein the step of extracting fungi comprises mechanically separating fungi from the compost by impinging the compost with air.

4. The method of claim 1 wherein the step of extracting fungi comprises mechanically separating fungi from the compost by impinging the compost with water.

5. The method of claim 1 wherein the step of extracting fungi comprises mechanically separating fungi from the compost by vibrating the compost.

6. The method of claim 1 wherein the step of extracting fungi comprises mechanically separating fungi from the compost ultrasonically.

7. The method of claim 1 wherein the step of extracting fungi comprises a liquid extraction process.

8. The method of claim 1 wherein the step of extracting fungi comprises filling a brewing bag with fungi-rich compost, placing the filled brewing bag in water for a time, separating the fungi from the compost, and then removing the brewing bag from the water.

9. The method of claim 8 further comprising providing a second compost-filled brewing bag, placing the second brewing bag in the water for a time, and separating the fungi from the compost in the second brewing bag, and then removing the second brewing bag from the water.

10. The method of claim 1 further comprising transporting the liquid extract to an application site prior to the step of adding water and nutrients to the liquid extract.

11. A method for sequestration of carbon, comprising the steps of:
creating a fungi-rich vegetative compost;
placing the fungi-rich vegetative compost in liquid;
extracting fungi from the compost to create a liquid extract;
and
applying the extract to the soil at the application site to facilitate the growth of fungi in the soil.

12. The method of claim 11 further comprising adding water and nutrients to the liquid extract to greatly increase the quantity of fungi in the liquid extract to create a fungi-rich extract.

13. The method of claim 12 further comprising transporting the liquid extract to an application site and of adding water and nutrients to the liquid extract.

14. The method of claim 13 wherein the step of adding water to the liquid extract occurs prior to applying the liquid extract to the soil.

15. An apparatus for manufacturing a carbon sequestration liquid extract, comprising:
a vessel for receiving water;
a compost introduction conveyor for adding compost to water contained within the vessel;
a compost removal conveyor for removing compost from the water;
a mechanical separator is positioned between the compost introduction conveyor and the compost removal conveyor for separating the fungi from the compost while the compost is in the water within the vessel to create a fungal-rich extract; and
an outlet for receiving the fungi-rich extract and outputting it for storage or delivery.
